(19) United States
(12) Patent Application Publication
(10) Pub. No.: US 2021/0038106 A1
(43) Pub. Date: Feb. 11, 2021

(54) MOBILE, WEARABLE EEG DEVICE WITH HIGH QUALITY SENSORS

(71) Applicant: THE TRUSTEES OF THE UNIVERSITY OF PENNSYLVANIA, Philadelphia, PA (US)

(72) Inventors: Arjun RAMAKRISHNAN, Lansdowne, PA (US); Michael L. PLATT, Philadelphia, PA (US); Naz BELKAYA, Philadelphia, PA (US); Rimjhim CHAUDHARY, Philadelphia, PA (US)

(21) Appl. No.: 17/016,290

(22) Filed: Sep. 9, 2020

Related U.S. Application Data
(63) Continuation-in-part of application No. PCT/US2019/021379, filed on Mar. 8, 2019.
(60) Provisional application No. 62/641,242, filed on Mar. 9, 2018.

(51) Int. Cl. A61B 5/0478; A61B 5/00

(52) U.S. Cl. CPC A61B 5/0478; A61B 5/6803; A61B 2562/164; A61B 2562/125; A61B 2562/06; A61B 2562/0285; A61B 2562/0209

(57) ABSTRACT

Provided are sensor units that include conductive segments disposed in a flexible sensing layer material, which sensor units are useful in EEG and other diagnostic applications. The sensor units are capable of maintaining electrical contact with a subject's skin in the absence of electrolytic gel, and provide robust signal collection with minimal signal degradation over time. Also provided are related methods of making the disclosed sensor units as well as methods of use.

### RELATED APPLICATIONS

This application is a continuation-in-part of international patent application PCT/US2019/021379, “Mobile, Wearable EEG Device With High Quality Sensors” (filed Mar. 8, 2019), which application claims priority to and the benefit of U.S. Application No. 62/641,242, “Mobile, Wearable, EEG Device With High Quality Nanowire Sensors” (filed Mar. 9, 2018), the entireties of which applications are incorporated herein by reference for any and all purposes.

### GOVERNMENT RIGHTS

This invention was made with government support under Grant Number 15-1519 awarded by the National Science Foundation. The government has certain rights in the invention.

### TECHNICAL FIELD

The present disclosure relates to the field of electroencephalography monitors and to the field of nanowires.

### BACKGROUND

Mobile, wearable, low-cost electro-encephalogram (EEG) devices that offer research grade signals can provide a wealth of information regarding the brain states of individuals. This knowledge can be effectively utilized to develop continuous healthcare monitoring applications, personalized treatment protocols in medicine, individual customer-based advertisements for marketing, and optimal training programs in sports.

Conventional EEG devices used in research and in the clinics for the last several decades are not conducive to mobile real world applications because these are wired devices that tether participants to a given location, limiting the activities that they can perform. Furthermore, these devices are not amenable for long-term use because electrodes in these devices typically require the use of an electrolytic gel to make contact with the scalp. Such a gel, however, dries out over time, thereby reducing signal quality and increasing discomfort due to dried salt deposits. Accordingly, there is a long-felt need in the art for improved EEG devices.

### SUMMARY

In meeting the long-felt needs described above, the present disclosure first provides a sensor unit, comprising a flexible sensing layer comprising a plurality of conductive segments disposed in a first matrix material, the flexible sensing layer having a first surface configured for placement against the skin of a subject and the flexible sensing layer having a second surface; a flexible support layer contacting the second surface of the flexible sensing layer, the flexible support layer comprising a second matrix material; and a current carrier in electronic communication with the conductive segments, the current carrier optionally extending through the flexible support layer, the current carrier being configured to carry a current to, from, or to and from the conductive segments.

Also provided are monitors, comprising: a headcovering comprising a plurality of devices according to the present disclosure, the head covering being configured to place the flexible sensing layers of at least some of the devices into contact with the skin of a subject.

Further provided are methods, comprising: with one or more sensor units according to the present disclosure, collecting one or more signals of neurological activity of a subject.

Additionally disclosed are methods of fabricating a sensor unit, comprising; atop a flexible support layer that comprises a second matrix, disposing a dispersion of conductive segments; placing a current carrier into electronic communication with at least some of the conductive segments; disposing a first matrix material atop the dispersion of conductive segments and at least some of the current carrier; and curing the first matrix material so as to form a flexible sensing layer that comprises at least some of the conductive segments, the current carrier being at least partially embedded within the flexible sensing layer, the flexible sensing layer being disposed atop the flexible support layer, and the current carrier extending through the flexible support layer.

Also provided are sensor units, comprising: a sensing portion that comprises a plurality of conductive segments; a conductive contact in electronic communication with the sensing portion; a substrate, the substrate supporting the conductive contact and the sensing portion; and a sealing material disposed so as to mask at least a portion of the substrate and at least a portion of the conductive contact while a portion of the sensing portion is exposed through the sealing material.

Additionally disclosed are devices, the device comprising: a plurality of sensor units, a sensor unit, comprising: a sensing portion that comprises a plurality of conductive segments; a conductive contact in electronic communication with the sensing portion; a substrate, the substrate supporting the conductive contact and the sensing portion; and a sealing material disposed so as to mask at least a portion of the substrate and at least a portion of the conductive contact while a portion of the sensing portion is exposed through the sealing material, and at least two of the plurality of sensor units being individually electronically addressable.

Further provided are methods, comprising: disposing a portion of conductive segments onto a substrate; encapsulating the portion of conductive segments in a first portion of a flexible material such that the conductive segments are present at a first surface of the portion of the first portion of the flexible material; contacting the conductive segments with a current carrier; and disposing a second portion of flexible material so as to encapsulate the current carrier within the first portion of flexible material and the second portion of flexible material.

Additionally provided are sensor units, comprising: a flexible material having disposed therein an amount of conductive segments, the conductive segments being present at a surface of the flexible material; a current carrier, the current carrier being in electrical communication with the conductive segments and the current carrier being at least partially encapsulated within the flexible material.

Also disclosed are sensor units, comprising: a flexible sensing layer comprising a plurality of conductive segments disposed in a first matrix material, the flexible sensing layer having a first surface configured for placement against a subject, the plurality of conductive segments optionally being characterized as a network, the plurality of conductive segments defining a first region of conductive segments that is exposed at the first surface, the plurality of conductive segments defining a second region of conductive segments that is enclosed within the flexible sensing layer, the second region of conductive segments being located at a distance, measuring along the first surface, from the first region of conductive segments; a current carrier in electronic communication with the conductive segments, the current carrier encapsulated within the flexible sensing layer, the current carrier being configured to carry a current to, from, or to and from the conductive segments.

Also provided are methods, comprising collecting a signal from a subject with a sensor unit according to the present disclosure

Further disclosed are methods, comprising: disposing a portion of conductive segments onto a substrate; encapsulating the portion of conductive segments in a first portion of a flexible material such that the conductive segments are present at a first surface of the portion of the first portion of the flexible material; contacting the conductive segments with a current carrier; and disposing a second portion of flexible material so as to encapsulate the current carrier within the first portion of flexible material and the second portion of flexible material.

### BRIEF DESCRIPTION OF THE DRAWINGS

For the purpose of illustrating the invention, there is shown in the drawings a form that is presently preferred; it being understood, however, that this invention is not limited to the precise arrangements and instrumentalities shown.

FIGS. 1A-1C provide an exemplary device. FIG. 1A provides an exemplary device with two partially visible AgNW (i.e., silver nanowire)-PDMS sensors (located behind the band of the cap and the forehead of the model). FIG. 1B provides an interior view of the cap showing three sensors according to the present disclosure (sensors are located the front of the cap). FIG. 1C provides an exemplary arrangement of sensor units according to the present disclosure; sensors can be disposed at the forehead with ground electrodes disposed at other positions.

FIG. 2 provides a baseball cap having sensors according to the present disclosure placed around the headband of the baseball cap such that the sensors would contact the forehead of the cap's wearer.

FIGS. 3A-3C provide input-output characterizations of exemplary sensors. FIG. 3A illustrates the sensor output when a 50 uV (peak-peak) signal was delivered, showing the near-zero lag of the sensor as the input and output traces essentially overlie one another. FIG. 3B illustrates that signal loss increased only slightly (factor of 0.02) with a greater than 10-fold decrease in input voltage (3 uV). FIG. 3C shows the ratio of the output to the input for two frequencies. FIG. 3D shows that signal loss barely increased (factor of 0.002) even with a 50-fold increase in signal frequency.

FIG. 4A and FIG. 4B provide comparisons between an exemplary silver nanowire sensor (AgNW) according to the present disclosure and an exemplary silver/silver chloride (Ag/AgCl) wet electrode, which is considered the “gold standard” for EEG measurements. FIG. 4A provides voltage fluctuations observed when eyes are open (spikes indicate blinks), the AgNW and Ag/AgCl traces are essentially identical to one another. FIG. 4B provides (top panel and lower left panel, showing magnified view of boxed region of top panel)) the voltage fluctuations (for disclosed and standard electrodes) when the subject's eyes were closed and also the power spectrum (lower right panel), with an increase in alpha power (8-12 Hz).

FIGS. 5A and 5B provide signal quality comparison over time. FIG. 5A shows that signals from standard Ag/AgCl sensors worsen over time as the contact gel dries up, whereas there is little to no signal degradation with use of the disclosed sensors (AgNW). FIG. 5B shows that the signals from two exemplary AgNW sensors remain highly correlated (>0.8), whereas the correlation between the AgNW and Ag/AgCl sensor reduces to zero over 70 min. FIG. 5C illustrates that a standard Ag/AgCl sensor picks up wire movement artifacts (noise, upper panel) and amplifier movement artifacts (noise, lower panel) when the gel dries up, whereas the disclosed AgNW sensors were robust to different noise artifacts.

FIG. 6 provides exemplary traces of signals with blink and eye movement artifact removal using Independent component analysis; as shown, artifact filtering was improved with three or more sensors than with two channels.

FIG. 7 provides a cross-sectional view of a device according to the present disclosure;

FIG. 8 provides a view of a device according to the present disclosure;

FIG. 9 provides engagement (theta/alpha EEG) during a user's exploration of a conference, as gathered by a device according to the present disclosure;

FIG. 10 provides a cross-sectional view of a device according to the present disclosure;

FIGS. 11A-11E provide an illustration of an exemplary device according to the present disclosure, with FIG. 11A providing an elevation view, FIG. 11B providing a top-down view, FIG. 11C providing a cross-sectional view, FIG. 11D providing a close-up view of the left-hand circled region in FIG. 11C, and FIG. 11E providing a close-up view of the right-hand circled region in FIG. 11C;

FIGS. 12A-12C provide an illustration of an exemplary mask used in the disclosed technology;

FIG. 13A provides a stepwise view of the fabrication of a plurality of devices according to the present disclosure;

FIG. 13B provides cross-sectional views of the steps shown in FIG. 13A; and

FIG. 13C provides a magnified view of certain portions of FIG. 13B.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps may be performed in any order. Any documents cited herein are incorporated by reference in their entireties for any and all purposes.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

In addition, the term “comprising” should be understood as having its standard, open-ended meaning, but also as encompassing “consisting” as well. For example, a device that comprises Part A and Part B may include parts in addition to Part A and Part B, but may also be formed only from Part A and Part B.

To fill the long-felt needs described above, provided are improve EEG devices; these devices can utilize nanosensor electrodes that provide high-grade signals. Signals collected by the disclosed electrodes can be amplified by a wireless amplifier, which can be read out using, e.g., an open-source hardware and software.

The disclosed sensors make use of a variety of conductive materials, e.g., silver nanowires (AgNW). The conductive materials can (though this is not a requirement) be disposed within a soft polymeric material, e.g., polydimethylsiloxane (PDMS). PDMS is considered especially suitable for biosensors because of its hydrophobic nature, biocompatibility, and mechanical properties that are similar to human soft tissue. In addition, the pliability of PDMS can help the sensor adhere to the skin, which improves skin-sensor contact leading to better signals. This property also reduces relative skin-electrode movement thereby minimizing motion artifacts.

A device according to the present disclosure can use one, two, or more such electrodes, which can be disposed on the subject's forehead. Signals from these sensors can be amplified and digitized using an amplifier.

Materials

To fabricate exemplary sensors according to the present disclosure, the following materials were used:

AgNW-L100™ in ethanol (www.acsmaterial.com/silvernanowire-500 mg-3249.html)

PDMS—Sylgard™ 184 kit

4-ch OpenBCI Cyton+OpenBCI software

EEG Device Designs

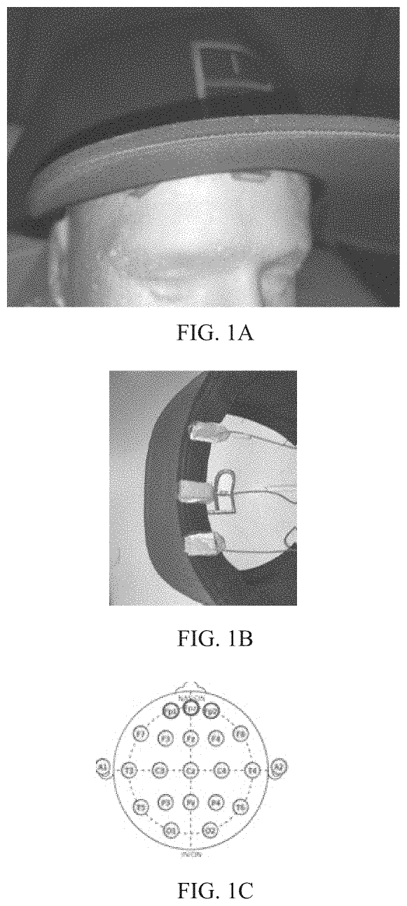

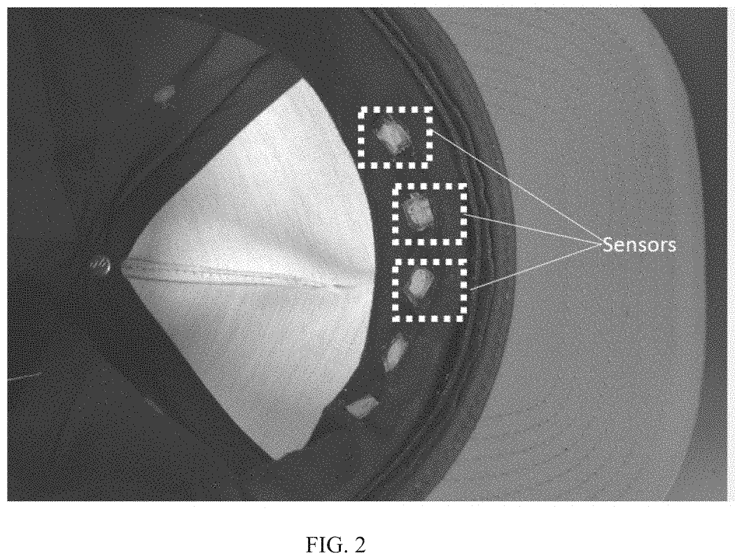

One exemplary design incorporated nanosensors in a baseball cap (FIG. 1A). This design included three AgNW sensors on the forehead (FIG. 1B) at Fp1, Fpz, and Fp2 based on a 10-20 EEG montage (FIG. 1C); the external ear (A2) was used as the ground. Three electrodes were used for removal of blink- and eye movement-related artifacts, the main source of noise at these locations. The signals from the AgNW sensors were amplified and digitized using an amplifier.

Nanosensor Fabrication

For fabrication, PDMS, PDMS curing agent, AgNW-ethanol solution, 4-inch Si wafer, aluminum heating cup, weight, scalpel, light duty lead wire, heater, vacuum chamber were used. One set of exemplary fabrication steps is described below:
Curing a desired thickness of PDMS on a Si surface.
A rectangle in the dimensions of the electrode to be made is cut and peeled off, leaving the Si wafer below exposed.
An AgNW-ethanol solution is drop-casted on the Si surface covering the exposed area with a thin layer. (Exemplary AgNWs were from ACS materials, e.g., https://www.acsmaterial.com/silver-nanowire-500 mg-3249.html).
After partial evaporation of the ethanol from the AgNW solution, the peeled wire tip of a light-duty lead wire was inserted on top of the solution and covered with another layer of AgNW which makes contact with the initial layer.
After the ethanol evaporates from the solution, a 10:1 ratio PDMS solution without any bubbles was poured over the exposed area until it is at the same level of thickness as the cured part.
The PDMS is cured, and then the relevant section of PDMS including the wire is cut and peeled off from the Si surface.

A schematic showing the different layers of an exemplary nanosensor is shown in FIG. 2B, along with a view of a prototype nanosensor.

Connecting the Sensor with an Amplifier and Deriving EEG Signals

Crimping pins were soldered onto the terminals of the light-duty lead wires and inserted into a 16-pole frame. The frame was attached to its male counterpart on the amplifier. Software was then used to read the raw EEG signals. EEG data was then exported in European Data format (EDF), opened and analyzed in Matlab using EEGLAB.

Results

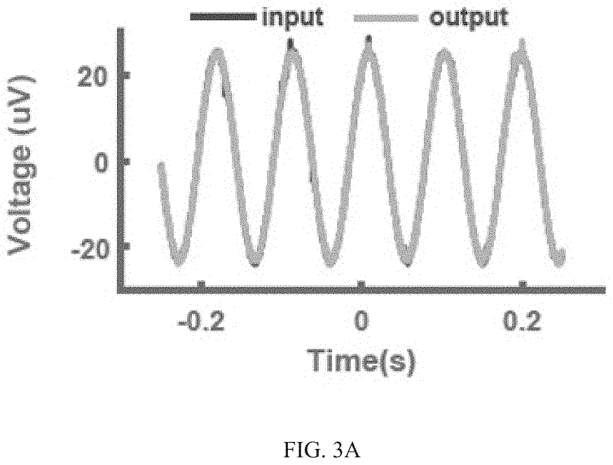

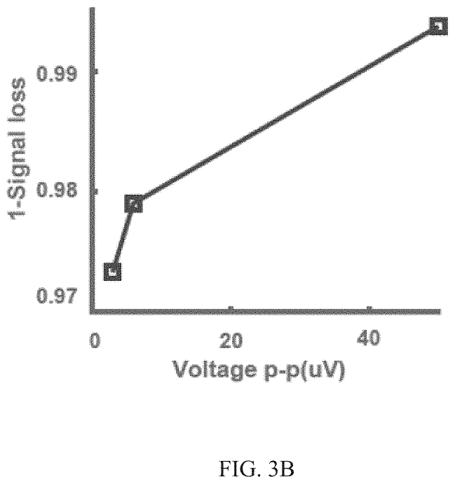

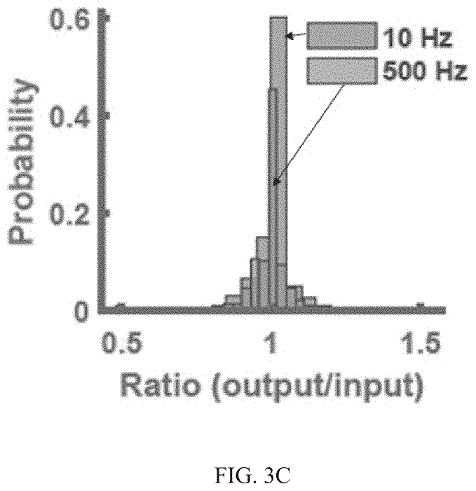

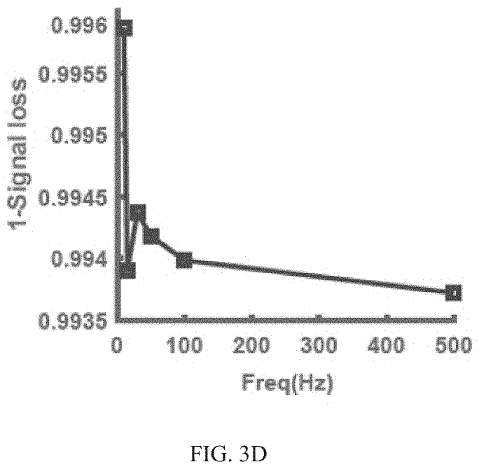

Input-output characterization of the nanowire sensor: To test the conductivity of the sensors for EEG signals, currents were administered at various voltages and frequencies that encompass EEG voltage and frequency range (FIG. 3). The input and the output signals were closely matched for an example voltage and frequency (FIG. 3A) suggesting very low loss. Signal loss increased slightly at very low voltages (FIG. 3B) and at very high frequencies (FIG. 3D). Overall, the new nanosensor displayed very low signal loss at EEG-level voltages and frequencies.

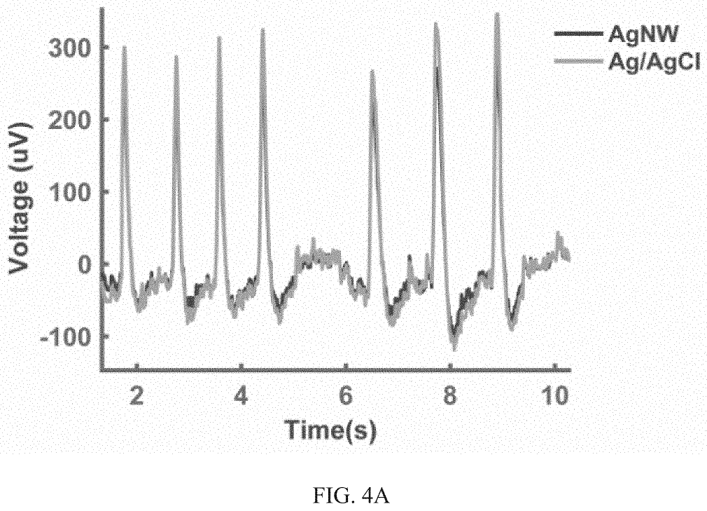

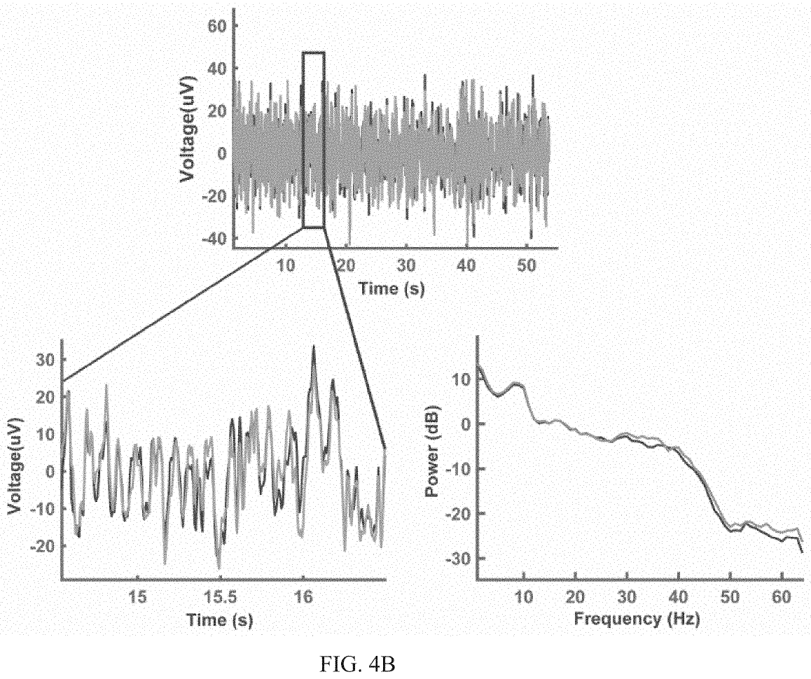

Comparison of the silver nanowire sensor with the standard EEG Ag/AgCl electrodes: Following characterization, an exemplary sensor (AgNW) was compared against standard Ag/AgCl EEG wet electrodes in a traditional EEG set-up. To do this, the two sensors were both placed at the same location on the forehead (Fp2). EEG signals were then picked up, amplified and transmitted using an amplifier. The received EEG signals were then examined during epochs when the participant's eyes were open (FIG. 4A) and when the eyes were closed (FIG. 4B). The signals from the new sensor were closely matched (r>0.9) in terms of voltage fluctuations (inset, left panel, FIG. 4B) and power content (inset, right panel, FIG. 4B), thus validating the quality of the nanosensor.

Long-term recording and movement artifacts: Wet electrodes typically use gel, which will eventually dry out, affecting signal quality over time. Because the disclosed sensors do not use gel, one may expect the signal quality to remain unaffected over time.

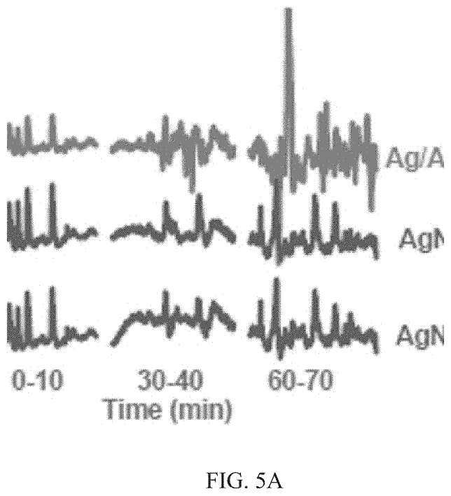

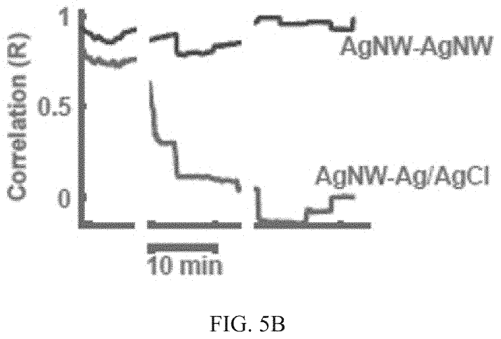

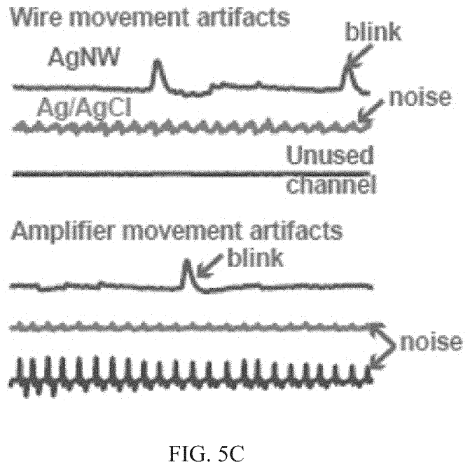

To examine this aspect of the disclosed devices, signal quality from a wet electrode and a nanosensor was monitored over a period of 70 minutes. Initially, the signal quality of the Ag/AgCl electrode matched that of the AgNW electrodes (0-10 min, FIG. 5A). However, over time, as the gel started to dry, the Ag/AgCl signal quality deteriorated (30-40 min and 60-70 min, FIG. 5A).

To visualize signal loss, the signal correlation between two AgNW sensors was compared with that between an Ag/AgCl and AgNW sensor (FIG. 5B). The two AgNW sensors remained highly correlated (r>0.85) over time. Reduction in Ag/AgCl signal quality decreased the latter correlation to zero within an hour (60-70 min, FIG. 5B).

In addition, when gel dries, Ag/AgCl wet electrodes are rendered vulnerable to noise artifacts. By contrast, AgNW sensors that do not use gel and that stick to the skin surface are be robust against such movement noise.

To illustrate this, movement noise was induced by moving the wire connecting the sensor to the amplifier (upper panel, FIG. 5C), and then by moving the entire device including the amplifier (lower panel, FIG. 5C). When the wire moved (upper panel, FIG. 5C), and when the amplifier moved (lower panel, FIG. 5C), these movements contaminated the EEG signal from the Ag/AgCl sensor. However, as predicted the AgNW sensors were robust to these sources of noise (FIG. 5C).

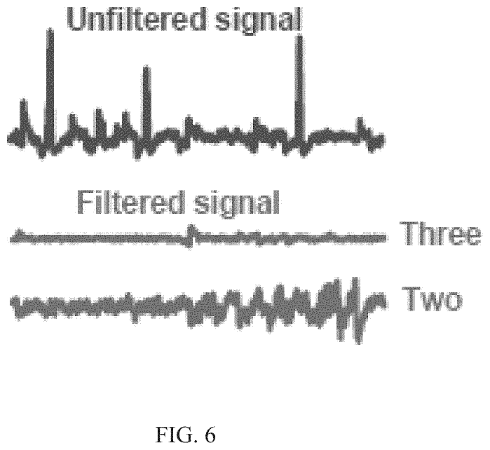

Blink and eye-movement artifact removal: Blinks and eye movements induce artifacts in EEG signal. Typically, blinks are removed using independent component analysis (ICA). Since this is a comparative analysis, at least two channels of information are required. ICA was performed using data from two AgNW channels and then using three AgNW channels (FIG. 6). Clearly, higher channel count improved noise elimination. With three channels, blinks and eye movements were almost entirely removed. These results suggest that three or more channels are required for good quality filtering.

Sensor signal collection was assessed in a few ways. First, signal to noise ratio was measured at different voltages and frequencies. A known input signal was fed to the sensor and the corresponding out signal was measured to characterize signal to noise ratio. Voltages spanning 10-50 uV (peak to peak) and frequencies spanning (0.1-500 Hz) were tested. The signal to noise ratio was greater than 97% for the lowest voltages and the highest frequencies.

A sensor-skin interface impedance was measured over time; this is a passive measure of the contact quality which directly affects signal quality of the physiological signals. Impedance measured in this way was compared with industry standard Ag/AgCl wet electrodes. It was found that not only was the impedance of the disclosed devices low (<15 kOhms), but the impedance also continued to remain low over long periods of time (120 minutes). By comparison, Ag/AgCl wet electrode impedance increased as the gel used with those wet electrodes dried up over time (>30 kOhms after 90 minutes). Voltage fluctuations and frequency content of the signal from these sensors were also compared with those obtained using the industry standard Ag/AgCl wet electrode, and it was found that the signals were highly correlated (r>0.8).

Additional Device Structure

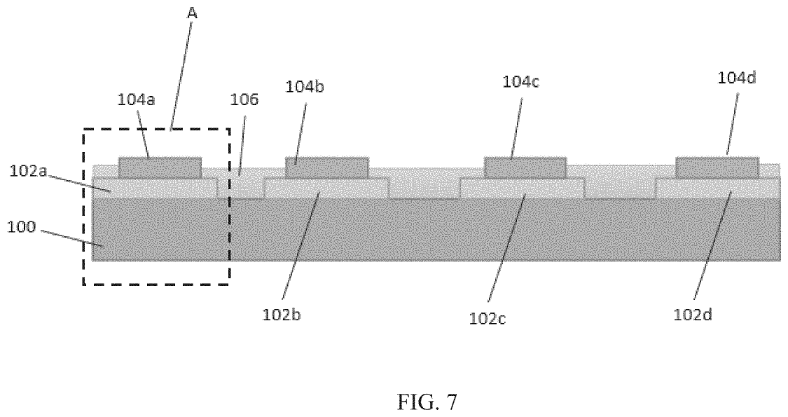

An exemplary embodiment is shown in FIG. 7. As shown in that FIG., a device can be formed in a layered fashion. A substrate 100 can be used to support conductive contacts (e.g., gold, copper, silver, titanium, a carbonaceous material) 102a, 102b, 102c, and 102d. Sensing portions 104a, 104b, 104c, and 104d can be formed atop the conductive contacts. A sealing material 106 can be disposed so as to cover and protect portions of the substrate 100, the contacts 102a-102d, and the sensing portions 104a-104d. A single sensor unit is shown by the dashed-in box A.

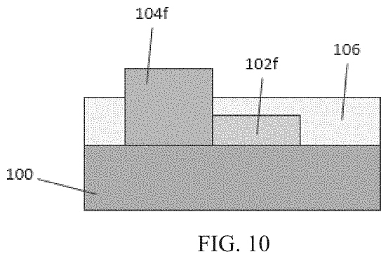

An alternative embodiment is shown in FIG. 10. As shown in that FIG., a device can be formed in a layered fashion. A substrate 100 can be used to support a conductive contact 102f The conductive contact 102f can be disposed such that it contacts the sensing portion (104f) and the substrate 100. Sealing material 106 can be disposed so as to cover and protect portions of the substrate 100, the contact 102f, and sensing portions 104f. As shown in FIG. 10, a sensing portion (1040 can contact the substrate (100) and also contact a conductive contact (1020, while also extending above the sealing material (106).

It should be understood that sensing portions can be formed directly on the substrate. As one example, silver nanowires can be deposited directly on a polyimide substrate. Conductive contacts can also be formed directly on the substrate, and can be in physical or electronic communication with the sensing portions. The sealing layer can, as described elsewhere herein, be deposited so as to cover the substrate and cover the conductive contacts, leaving exposed the sensing portions.

A sensing portion (e.g., 104a in FIG. 7, 104f in FIG. 10) can comprise conductive segments, such as nanowires, e.g., silver nanowires, but can also include, e.g., gold, copper, and/or platinum nanowires and/or nanoparticles. Carbon nanotubes, graphene portions, and the like are also suitable conductive segments. Conductive segments can be disposed in a matrix material (e.g., a polymeric material), but this is not a requirement, as conductive segments can be “bare” or otherwise free of being embedded in a matrix material.

The sealing material 106 can be a flexible material, and can be a material that is comfortable to touch to the skin. Polydimethylsiloxane (PDMS) (as well as other siloxanes), polymethyl methacrylate (PMMA), parylene, polyimide, flexible polyethylene terephthalate (PET), poly(3,4-ethylenedioxythiophene) (PEDOT), polyurethane (PU), and other such materials are all suitable sealing layer materials.

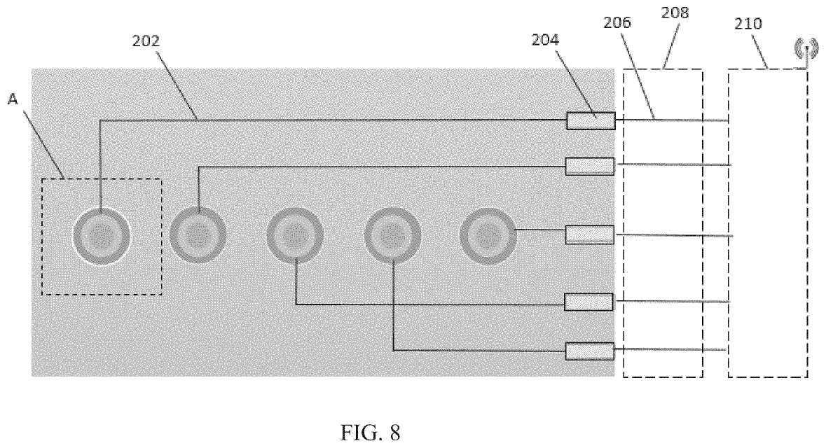

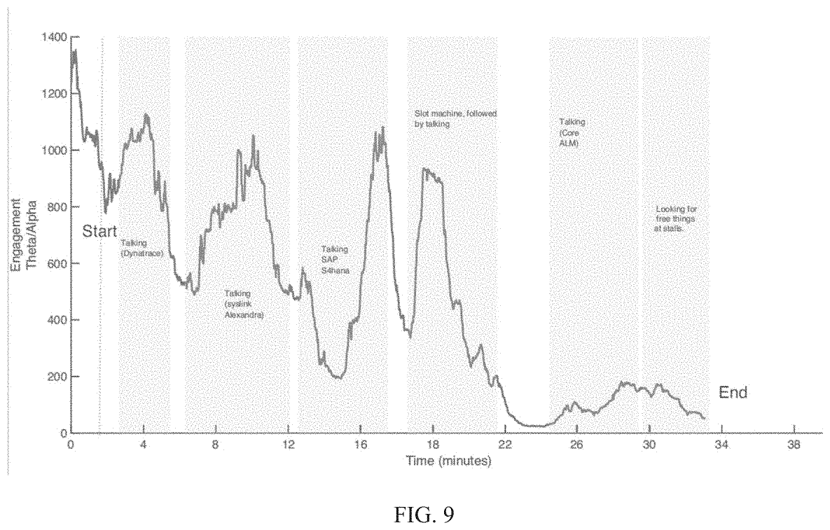

An exemplary process for fabricating a device such as that shown in FIG. 7 (and/or in FIG. 10) is as follows:
A layer/film (e.g., Kapton™ or other polyimide; other materials are also suitable) is used as a substrate (100).
A shadow mask (e.g, made of stainless steel), is prepared to facilitate deposition of conductive contacts (102a, 102b) of the desired size, shape, and spacing.
In one exemplary deposition for gold, a 10 nm thick layer of titanium is deposited followed by a 200 nm thick film of gold. Metal deposition can be done under a vacuum, and can be done by evaporating the metal using an electron beam evaporator.
Another shadow mask can be used to deposit the conductive segments, which are then cast through the mask, e.g., via drop casting. A layer of the conductive segments can be, e.g., from 1000 to 2000 nm in thickness, although this is not a rule or requirement. The shadow mask can be used to pattern conductive segments in a periodic pattern, e.g., in evenly-spaced dots. A periodic pattern is not, however, a rule or a requirement.
Following deposition of the conductive segments, the sealing layer (e.g., in a curable or harden-able liquid form) is applied. As one example, a PDMS solution is applied until the desired thickness (e.g., 1000 nm) is achieved. The sealing layer can be applied such that it covers the exposed substrate while also leaving the conductive segments exposed. In this way, a user is contacted by the sensing portion (e.g., 104a) and the sealing layer (106).
FIG. 8 provides a top view of a device according to the present disclosure. As shown, a sensor unit (again shown by the dashed-in box A, as in FIG. 7) can be connected via conductor 202 to a contact 204. A sensor unit can be connected directly to an amplifier 210, without any intervening signal processing. In some embodiments, signal collected by a sensor unit can undergo processing at processing train 208 (optional) before being communicated to amplifier 210.
FIG. 9 provides engagement (theta/alpha EEG, shown by solid trace) during a user's exploration of a SAP TECHED conference, as gathered by a device according to the present disclosure. The bouts of activities (texting, talking, etc.) that participants engaged in as they moved around the conference show floor are overlaid in grey. Unmarked periods indicate the participant is walking. Entry to the conference hall is indicated by a dashed line. As shown, various activities resulted in various levels of engagement by the subject.

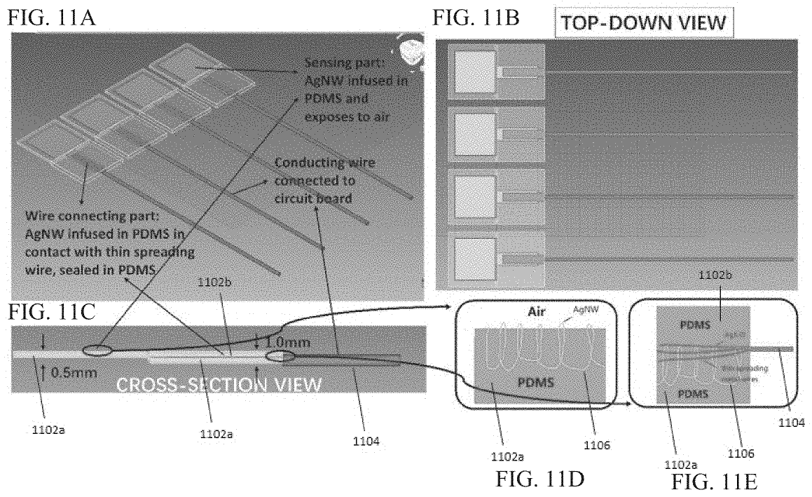

Additional disclosure is provided in FIGS. 11A-11E, FIGS. 12A-12C, and FIGS. 13A-13B.

FIG. 11A provides an elevation view of a sensor according to the present disclosure. As shown, a sensor may present a sensing potion (that comprises conducive segments disposed (e.g., via infusion into) on or within a flexible material, e.g., PDMS. (It should be understood that although PDMS is used as an exemplary flexible material herein, PDMS is illustrative only and the present disclosure is not limited to the use of PDMS.) A current carrier can place the sensing portion into electronic communication with the environment exterior to the sensor, e.g., by placing the sensing portion into electronic communication with a monitor, processor, or other module or instrument that receives a signal collected by the sensing portion. (As shown, the current collector can place the sensing portion into electronic communication with a circuit board.) The current collector can be embedded in the flexible material, e.g., embedded within a single portion of the flexible material, but can also be disposed between two portions of flexible material, e.g., disposed between two portions of flexible material, such as PDMS. In such an embodiment, the two portions of PDMS can be sealed, bonded, or otherwise adhered to one another, although this is not a requirement. The current collector can be bonded or adhered to the flexible material.

As shown in FIG. 11B (top-down view), multiple sensing portions can be arranged in parallel or otherwise arrayed, with each sensing portion being individually addressable. As shown in FIG. 11b, a given sensing portion can be associated with its own discrete portion of flexible material, but this is not a requirement, as a portion of flexible material can be associated with a plurality of sensing portions. As but one example, a single portion of flexible material can support two, three, or more sensing portions.

FIG. 11C provides a cross-sectional view of a sensor unit according to FIG. 11A. As shown, the sensing unit can include a portion (1102a) of flexible material that has therein an amount of conductive segments, e.g., silver nanowires (AgNW). The flexible material 1102a can have a kink or bend in it, as shown. The sensor unit can also include a portion (1102b) of flexible material, which portion 1102b can be superposed on portion 1102a, as shown. In this way, a sensor unit can be of variable thickness along its length; as shown, the relatively thin portion of the sensor unit (defined by the thickness of 1102a) is 0.5 mm, and the relatively thicker portion of the sensor unit (defined by the thickness of 1102a and the thickness of 1102b) is on the order of 1.0 mm, as 1102b is superposed on 1102a. A sensor unit can also include current carrier 1104.

As shown in FIG. 11D (a close-up of portion 1102a, circled in FIG. 11C), a portion of conductive segments 1106 (AgNW, in this instance) disposed within portion 1102a. FIG. 11E is a close-up of the other circled portion in FIG. 11C. As shown in FIG. 11E, the current collector 1104 can be a braid or other collection of wires, which wires are untwisted or otherwise fanned out so as to effect improved electrical communication with the conductive segments 1106. Also as shown in FIG. 11E, the current collector 1104 and the conductive segments 1106 can be disposed between portions 1102a and 1102b of the flexible material. It should be understood, however, that the current collector and the conductive segments can be disposed within a single, monolithic portion of flexible material.

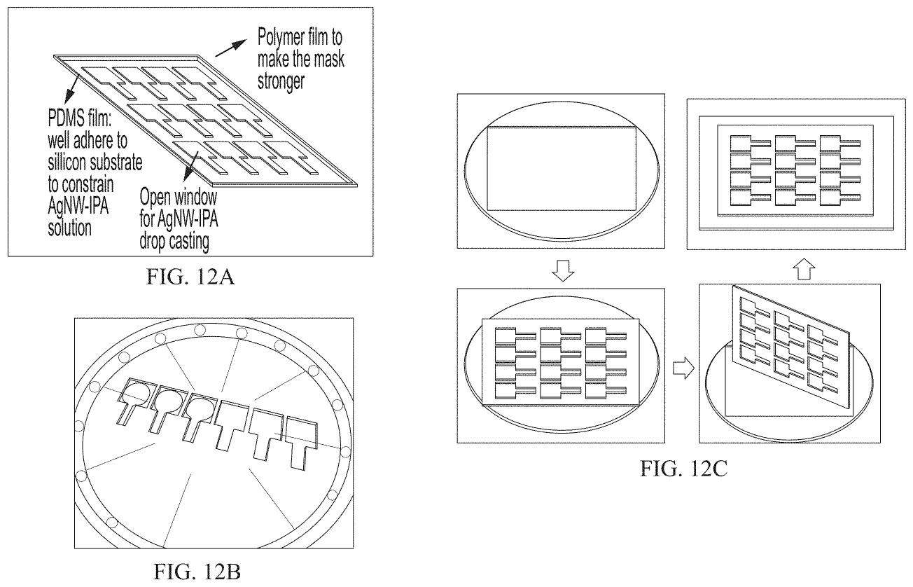

As shown, a sensor can include three parts: the sensing part, the wire connecting part, and the connecting wire. At the sensing part, the thin silver nanowire (AgNW) network is infused in PDMS, and is right at the surface of the PDMS sheet, as shown in FIG. 12D. This part is designed to be in contact with skin and detects the electrical signal with AgNW that come out from the PDMS matrix.

The AgNW network bends to the center of the PDMS in the wire connecting part, in contact with thin spreading metal wires emanating from a small gauge wire, as indicated in FIG. 12E. The connection structure between AgNW network and metal wires is encapsulated in the PDMS. The metal wires extend out from the PDMS matrix and are connected to the signal processing circuit. In this way the metal wires are easier to be handled, and the thickness of the sensor sheet can be reduced to appx. 1 mm, which can result in lower material cost and higher flexibility of the sensor.

FIGS. 12A-12C provide an illustration of an exemplary mask used in the disclosed technology. As shown in FIG. 12A, a user can use as a mask a flexible material (e.g., PDMS) in which open windows are defined to contain a solution of conductive segments. The mask can be supported by a further support material, e.g., a polyethylene film or other polymer) to support the mask and provide rigidity, if needed. FIG. 12B provides an illustrative image of windows defined in a mask. FIG. 12C provide a stepwise view of mask usage; as shown, a user can define windows in the flexible material, which flexible material can then be used as a mask for deposition of conductive segments.

As an example, an AgNW network was disposed onto a Si surface with drop-cast and evaporation of AgNW-IPA suspension solution, followed by PDMS infiltration and peeling off to fabricate the EEG sensor.

To assist this solution-based casting process in a scalable way, a drop-cast mask was used. The drop-cast mask included a layer of thin PDMS sheet (e.g., 0.5 mm thick) with window patterns on it and a layer of polyester film, and these two parts are attached together, as shown in FIGS. 12A-12C, which demonstrate the process flow of making the drop-cast mask:

1. A layer of uniform PDMS sheet is first made by spin-coating PDMS solution (base:cure=10:1) on a Si wafer, cure at 95 Deg.C. for 30 min.

2. After that, a polyester film was cut with a laser cutter to open the windows for drop-cast, and then stuck to the PDMS sheet on the Si wafer, followed by PDMS peeling off from the Si wafer.

3. Finally, the whole mask is placed on a rubber sheet, and the window patterns on the PDMS sheet were cut out with a cutting die of certain shapes.

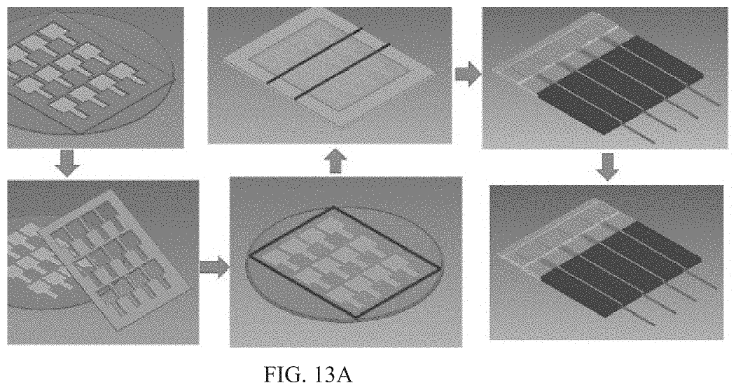

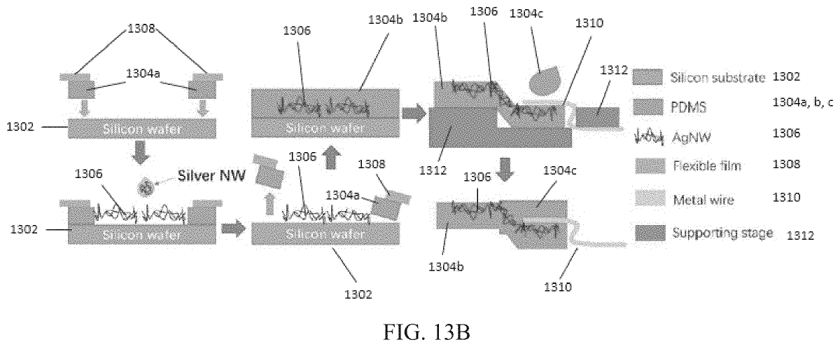

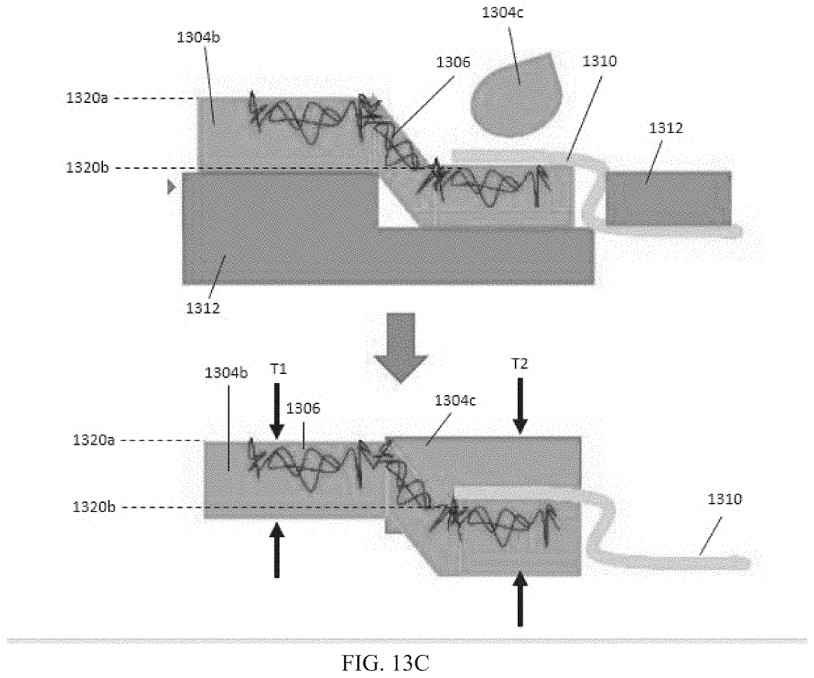

FIG. 13A provides a step-wise view of the fabrication of a plurality of devices according to the present disclosure. FIG. 13B provides cross-sectional views of the steps shown in FIG. 13A.

By reference to FIGS. 13A-13B and by reference to exemplary, non-limiting materials, an exemplary fabrication process

1. A drop-casting mask was gently pressed onto the smooth surface of a Si wafer to let the PDMS sheet seal to the Si surface.

2. After that, AgNW-IPA solution was casted onto the Si surface through each window on the mask, which are constrained within the window area by the PDMS sheet. The IPA evaporates over time (˜15 min), leaving the AgNW network free-standing on the Si surface. This drop-cast process was repeated to increase the AgNW density, although this was not a requirement.3

3. The drop-cast mask is lifted off from the Si surface. PDMS solution (base:cure=10:1) is spun coated onto the Si wafer covering the free-standing AgNW network, cure at 95 deg. C. in an oven for 30 min, then the PDMS layer is peeled off from the Si wafer.

4. The PDMS layer infused with AgNW patterns is placed on a rubber sheet and cut with a cutting die into individual pieces. After that, these PDMS pieces are placed onto a substrate with a stepped structure, with the sensing part sitting on the higher side of the step and the AgNW side facing up. Spreading thin metal wires from small gauge (e.g., 30 AWG) are then placed on top of the wire connecting parts of the PDMS pieces sitting on the lower side of the step, in contact with the AgNW patterns. Kapton tapes are pressed onto the metal wire-AgNW connection and hold them together. (The presence of a tape or sealant is optional, but can be useful to maintain the physical connection between the current collector/wire and the conductive segments.)

5. A further PDMS solution (base:cure=10:1) was poured onto the step structure, and under gravitation and surface tension force, the solution fills up the lower side of the step and flattens the step, encapsulating the metal wire-AgNW connection within the PDMS layer. By controlling the PDMS (encapsulating) solution applied at this stage, the encapsulation layer will not cover the sensing part of the AgNW pattern, the small amount of redundant PDMS will just flow away from two side of the step structure.

6. After another 95 deg. C. cure for 30 min, the PDMS sheets with the relevant AgNW patterns and metal wires are peeled off from the step structure and were ready for further incorporation into a headband or other device.

FIG. 13b provides cutaway views of the steps shown in FIG. 13a. As shown, one can begin with a substrate 1302 (e.g., a silicon wafer) on which a mask (1304a) is disposed, with the mask defining at least one window therethrough. The mask can be formed of PDMS, and the mask can also include a support (1308), which support can be a flexible film, such as Kapton™ or other polymeric or even metallic film. As shown, a user can dispose an amount of a solution that comprises conductive segments (1306), which segments can be, e.g., silver nanowires (AgNW), into the window formed in the mask. The carrier material (e.g., alcohol, water or other solvent) of the solution can be evaporated so as to leave behind a portion of the conductive segments that conforms (at least partially) to the shape of the window formed in the mask; the conductive segments can also be disposed directly on substrate 1302. As shown, the mask can be removed, and an amount (1306b) of a flexible material (e.g., PDMS, as one example) can be disposed atop the portion of conductive segments 1306. As shown, this can result in a portion (e.g., a layer) of flexible material 1304b configured such that at least some of the conductive segments are disposed at the surface (or nearby to the surface) of the flexible material that is closes to the substrate 1302.

The portion 1304b of flexible material (with the conductive segments 1306 wherein) can be transferred to a stepped supporting stage 1312. As shown, the flexible material can be transferred such that the surface (referred to here, for convenience, as the upper surface) of the flexible material that bears the conductive segments 1306 is exposed. A current collector 1310 (e.g., a metal wire) can be contacted to the upper surface of the flexible layer. Although not shown in FIGS. 13A-13C, the current collector can be optionally secured to the upper surface, e.g., via tape or via a sealant/adhesive. Although not shown in FIGS. 13A-13C, additional conductive segments (e.g., AgNW) can be disposed on the current carrier so as to “sandwich” the current carrier between portions of conductive segments. This is not a requirement, however.

A further portion 1304c of flexible material can be disposed atop the current collector so as to encapsulate the current collector between portions 1304c and 1304b. Portions 1304b and 1304c can be of the same flexible material (e.g., PDMS), but this is not a requirement, as portions 1304b and 1304c can differ from one another in terms of composition. As shown, portion 1304c can encapsulate the current carrier and then become even or flush (though this is not required) with portion 1304b such that the final sensor unit has a thickness in one region that is the thickness of region 1304b, and a thickness in another region that is the combined thickness of regions 1304b and 1304c.

As an example (and by reference to FIG. 13c), a sensor unit can define a thickness T1 at a region that is configured for contact with a subject (i.e., where conductive segments are available at a surface to be placed into contact with a subject) and a thickness T2 at a region where the current carrier is present. The transition between thickness T1 and T2 can be a step-wise transition, but can also be gradual in nature. The ratio between T1 to T2 can be from 1:1.01 to 1:10, from 1:1.1 to 1:5, from 1:1.2 to 1:2, and all intermediate values.

Thus, fabrication can use a drop-cast mask to constrain the AgNW-IPA and assist AgNW solution-based printing, in place of directly cutting out pattern a on PDMS on Si wafer. This process avoids mechanical cutting. Moreover, the drop-cast mask is reusable.

### EXEMPLARY EMBODIMENTS

The following embodiments are exemplary only and do not limit the scope of the present disclosure or the appended claims.

Embodiment 1. A sensor unit, comprising: a flexible sensing layer comprising a plurality of conductive segments disposed in a first matrix material, the flexible sensing layer having a first surface configured for placement against the skin of a subject and the flexible sensing layer having a second surface; a flexible support layer contacting the second surface of the flexible sensing layer, the flexible support layer comprising a second matrix material; and a current carrier in electronic communication with the conductive segments, the current carrier optionally extending through the flexible support layer, the current carrier being configured to carry a current to, from, or to and from the conductive segments.

The flexible sensing layer can have a thickness in the range of from about 0.2 to about 2 mm, in some embodiments.

Embodiment 2. The sensor unit of Embodiment 1, wherein the current carrier is at least partially embedded in the flexible sensing layer.

Embodiment 3. The sensor unit of any of Embodiments 1-2, wherein the current carrier is characterized as being a wire.

Embodiment 4. The sensor unit of any of Embodiments 1-3, wherein the conductive segments comprise one or more metals, graphene, carbon nanotubes, or any combination thereof. Single- and multi-wall carbon nanotubes can be used, as can be other carbonaceous materials.

Conductive segments can be in the form of, e.g., nanowires. A nanowire can have a cross-sectional dimension (e.g., a diameter) in the range of from, e.g., about 1 to about 1000 nm, or even from about 5 to about 500 nm.

Nanowires having an average diameter in the range of from about 50 to about 300 nm are considered suitable. As described elsewhere herein, a nanowire can be metallic; gold and silver nanowires are considered especially suitable. A nanowire can have a length in the range of, e.g., from about 1 to about 1000 micrometer, or even from about 100 to about 200 micrometers.

A conductive segment can be a nanowire, but can also be of another nanostructure, including carbonaceous and metallic nanostructures such as nanofoams, nanofibers, nanoflakes, nanoparticles, nanoshells, quantum dots, and the like, as well as combinations of nanostructures. A device according to the present disclosure can include as conductive segments one, two, or more types of materials, e.g., nanowires and graphene platelets.

Without being bound to any particular theory, at least some the conductive segments disposed in the flexible sensing layer can be in contact with one another; such contact gives rise to a network (continuous or semi-continuous) of such segments. The network can be termed a nanomesh or even a “mat” of the conductive segments.

The conductive segments can be disposed uniformly/homogeneously (or nearly so) within the first matrix material. Put another way, the density of the conductive segments can be uniform through the thickness of the first matrix material, although this is not a rule or a requirement.

Embodiment 5. The sensor unit of Embodiment 4, wherein the conductive segments comprise one or more metals, e.g., silver or gold. Conductive segments can comprise, e.g., metallic nanostructures or a combination of metallic nanostructures. Silver nanowires are considered especially suitable, but are not the only suitable conductive segments.

Embodiment 6. The sensor unit of Embodiment 5, wherein the conductive segments comprise one or more metallic nanowires.

Embodiment 7. The sensor unit of Embodiment 6, wherein the conductive segments comprise gold nanowires, silver nanowires, or any combination thereof.

Conductive segments (whether metallic nanowires or other materials) can be bare or coating-free, but can also include a coating or functionalization. Salts (e.g., AgCl, NaCl, and the like) can be used as coatings, as one example. A conductive segment can also undergo a surface treatment, e.g., treatment by an oxidative plasma.

Before, during, or after incorporation of conductive segments into the first matrix material, the first matrix material can be stretched, bent, folded, wrinkled, or otherwise physically manipulated. Without being bound to any particular theory, such manipulation can improve the incorporation of the conductive segments into the first matrix material, e.g., increasing the physical density of the conductive segments in the first matrix material.

With respect to the conductive segments, the conductive segments can define from 0.1 to 99% of the overall volume of the volume of the first matrix material and the conductive segments.

It should be understood that any embodiment of the disclosed sensor units can include one or more surface features that engage with a user. Such surface features can include, e.g., a ridge, a bump, a cone, a pyramid, a “soft needle” (e.g., a soft bristle, fiber, or other protrusion extending from the sensor unit), and the like. Such features can facilitate suitable contact between the device and the user.

Embodiment 8. The sensor unit of any of Embodiments 6-7, wherein the metallic nanowires have a diameter in the range of from about 1 to about 1000 nm, e.g., from about 1 to about 1000 nm, from about 5 to about 750 nm, from about 10 to about 500 nm, or even from about 20 to about 200 nm.

Embodiment 9. The sensor unit of any of Embodiments 6-8, wherein the metallic nanowires have a length in the range of from about 1 to about 1000 micrometers, e.g., from about 1 to about 1000 nm, from about 5 to about 750 nm, from about 10 to about 500 nm, or even from about 20 to about 200 nm.

Embodiment 10. The sensor unit of any of Embodiments 1-9, wherein one or both of the first matrix material and the second matrix material is characterized as polymeric.

The first matrix material is suitably a material that is high-friction or “tacky” to the touch, as such materials are particularly well-suited to maintaining their position on a subject's skin without excessive sliding.

Embodiment 11. The sensor unit of Embodiment 10, wherein one or both of the first matrix material and the second matrix material is characterized as a polysiloxane and/or a silicon(e) elastomer.

Embodiment 12. The sensor unit of Embodiment 7, wherein one or both of the first matrix material and the second matrix material comprises polydimethylsiloxane (PDMS).

Embodiment 13. The sensor unit of any of Embodiments 1-8, wherein the first matrix material is characterized as being hydrophobic. Without being bound to any particular theory, a material that does not react with a subject's skin is considered especially suitable.

Embodiment 14. The sensor unit of any of Embodiments 1-9, wherein the sensor unit is capable of maintaining electrical contact with a subject's skin in the absence of an electrolytic material disposed between the flexible sensing layer of the sensor unit and the subject's skin.

Again without being bound to any particular theory, this can be accomplished by direct contact between the conductive segments in the flexible sensing layer and the subject's skin. The matrix material of the flexible material (e.g., PDMS) can be “tacky” or high-friction, which qualities can help the material maintain contact with the subject's skin.

Embodiment 15. The sensor unit of any of Embodiments 1-14, wherein the sensor unit's collection of an electrical signal from a subject is substantially unaffected by movement artifacts (e.g., current carrier movement, amplifier movement, or even subject movement), in the absence of an electrolytic material disposed between the flexible sensing layer of the sensor unit and the subject's skin.

As shown in FIG. 5C, sensors according to the present disclosure are able to collect signals from a subject with little to no change in the signal as a result of wire or amplifier movement.

It should also be understood that the disclosed sensors are capable of collecting consistent signals from a subject over an extended period of time (e.g., 30, 60, or even more minutes) in the absence of an electrolytic material between the sensor and the user's skin, i.e., without degradation of the signal during that period of time.

This stands in contrast to existing Ag/AgCl electrodes, which electrodes require the use of an electrolytic gel that, over time, dries out and results in signal degradation. This is shown by, e.g., FIG. 5, which shows the degradation of the signal collected by an Ag/AgCl electrode arrangement (with an electrolyte gel between the electrode and the subject) over 60 minutes.

Embodiment 16. The sensor unit of any of Embodiments 1-15, wherein noise in the sensor unit's collection of an electrical signal from a subject is substantially unaffected by movement artifacts, in the absence of an electrolytic material disposed between the flexible sensing layer of the sensor unit and the subject's skin.

Embodiment 17. The sensor unit of any of Embodiments 1-16, wherein the sensor is capable of maintaining electrical contact with a subject's skin at a hairy region of the scalp, in the absence of an electrolytic material disposed between the flexible sensing layer of the sensor unit and the subject's skin. Without being bound to any particular theory, the flexibility and/or tackiness of the disclosed sensors allows for this improved performance.

Embodiment 18. A monitor, comprising: a headcovering comprising a plurality of devices according to any of Embodiments 1-17, the head covering being configured to place the flexible sensing layers of at least some of the devices into contact with the skin of a subject.

Embodiment 19. The monitor of Embodiment 18, further comprising an amplifier in electronic communication with one or more of the plurality of devices. It should be understood that an amplifier can be separate from the device and/or the head covering. In some embodiments, however, the amplifier can be incorporated into the device or into the head covering.

Embodiment 20. The monitor of Embodiment 19, further comprising a filtration train configured to filter one or more signals collected by one or more of the plurality of devices.

Embodiment 21. The monitor of Embodiment 20, wherein one or more signals are of neurological activity of the subject.

Embodiment 22. The monitor of any of Embodiments 18-21, further comprising a portable power source in electronic communication with the amplifier. Such portable power sources include, e.g., batteries, solar cells, and the like.

Embodiment 23. The monitor of any of Embodiments 18-22, further comprising a display configured to display a signal of neurological activity of a subject that is collected by one or more of the plurality of devices.

Embodiment 24. The monitor of any of Embodiments 18-23, wherein the head covering is characterized as a hat.

Embodiment 25. The monitor of any of Embodiments 18-24, wherein the monitor is characterized as being portable.

Embodiment 26. A method, comprising: with one or more sensor units according to any of Embodiments 1-17, collecting one or more signals of neurological activity of a subject. The disclosed sensor units can be used in, e.g., EEG measurements and other medical diagnostic applications, in sports and daily routine.

Embodiment 27. The method of Embodiment 26, wherein the flexible sensing layers of the one or more sensor units contact the skin of the subject.

Embodiment 28. The method of any of Embodiments 26-27, further comprising removing one or more artifacts from one or more of the signals.

Embodiment 29. The method of any of Embodiments 26-28, further comprising filtering one or more of the signals.

Embodiment 30. The method of any of Embodiments 26-29, further comprising correlating one or more of the signals to an activity of the subject, to a stimulus received by the subject, or any combination thereof.

Embodiment 31. A method of fabricating a sensor unit, comprising: atop a flexible support layer that comprises a second matrix, disposing a dispersion of conductive segments; placing a current carrier into electronic communication with at least some of the conductive segments; disposing a first matrix material atop the dispersion of conductive segments and at least some of the current carrier; and curing the first matrix material so as to form a flexible sensing layer that comprises at least some of the conductive segments, the current carrier being at least partially embedded within the flexible sensing layer, the flexible sensing layer being disposed atop the flexible support layer, and the current carrier extending through the flexible support layer.

Suitable first and second matrix materials are provided elsewhere herein, and can include, e.g., PDMS. Similarly, suitable current carriers include, e.g., wires. Suitable conductive segments are also described elsewhere herein. The disclosed methods can be used, e.g., to fabricate a sensor unit according to any of Embodiments 1-17.

Curing of a matrix material can be effected by heat, UV illumination, or other methods known in the art.

Embodiment 32. The method of Embodiment 31, the method being performed so as to give rise to one or more sensor units according to any of Embodiments 1-14.

Embodiment 33. The method of any of Embodiments 31-32, wherein the flexible support layer is disposed atop a template substrate. Suitable template substrates include, e.g., Si, SiO₂, and the like.

Embodiment 34. The method of Embodiment 33, further comprising freeing the flexible support layer from the template substrate. This can be accomplished by, e.g., peeling.

Embodiment 35. The method of any of Embodiments 31-34, further comprising placing the sensor unit in a head covering.

Embodiment 36. A sensor unit, comprising: a sensing portion that comprises a plurality of conductive segments; a conductive contact in electronic communication with the sensing portion; a substrate, the substrate supporting the conductive contact and the sensing portion; and a sealing material disposed so as to mask at least a portion of the substrate and at least a portion of the conductive contact while a portion of the sensing portion is exposed through the sealing material.

Embodiment 37. The sensor unit of Embodiment 36, wherein the sensing portion contacts the substrate. In some embodiments, e.g., FIG. 7, the sensing portion does not contact the substrate.

A sensing portion can present a flat surface, e.g., a flat-topped cylinder or other formation. A sensing portion can also present a non-flat surface, e.g., a rippled, wrinkled, or otherwise uneven surface. In this way, a sensing portion can conform to the user.

Embodiment 38. The sensor unit of any one of Embodiments 36-37, wherein the conductive contact is in physical contact with the sensing portion and the substrate. As an example, a conductive contact can be formed on the substrate such that the conductive contact touches the substrate and also touches the sensing portion. Such an embodiment is provided in FIG. 10, in which the conductive contact 102f contacts the substrate 100 and also the sensing portion 104f As shown in FIG. 10, the sealing material 106 can be such that the sensing portion 104f is exposed above the sealing material, which the sealing material covers the substrate 100 and conductive contact 102f.

The sensing portion can be “taller” than the conductive contact in order that the sensing portion extends through and above the sealing layer, while the conductive contact remains covered by the sealing layer. One example of this is shown in FIG. 7, which depicts a sensing portion (104a) being “taller” than the conductive contact (102a) that is associated with that sensing portion.

Embodiment 39. The sensor unit of any one of Embodiments 36-38, wherein the conductive contact is disposed between the sensing portion and the substrate. One such example is shown in FIG. 7.

Embodiment 40. The sensor unit of any one of Embodiments 36-39, wherein a conductive segment comprises a metal nanowire, a carbon nanotube, a graphene portion, of any combination thereof. Silver nanowires are considered especially suitable as conductive segments.

Embodiment 41. The sensor unit of any one of Embodiments 36-40, wherein the substrate comprises a polyimide. Other materials, including flexible materials, are also considered suitable.

Embodiment 42. The sensor unit of any one of Embodiments 36-41, wherein the sealing material comprises PDMS, PMMA, parylene, polyimide, PET, poly(3,4-ethylenedioxythiophene); polyurethane, or any combination thereof.

Embodiment 43. The sensor unit of any one of Embodiments 36-43, wherein the plurality of conductive segments are disposed in a matrix material.

Embodiment 44. The sensor unit of Embodiment 43, wherein the matrix material comprises a polymeric material. PDMS is one suitable polymeric material, although others are also suitable.

Embodiment 45. The sensor unit of any one of Embodiments 36-43, wherein the sensor unit is in electronic communication with an amplifier.

Embodiment 46. A device, the device comprising: a plurality of sensor units, with a sensor unit comprising: a sensing portion that comprises a plurality of conductive segments; a conductive contact in electronic communication with the sensing portion; a substrate, the substrate supporting the conductive contact and the sensing portion; and a sealing material disposed so as to mask at least a portion of the substrate and at least a portion of the conductive contact while a portion of the sensing portion is exposed through the sealing material, and at least two of the plurality of sensor units being individually electronically addressable. As described elsewhere herein, a user can thus utilize individual sensing portions to collect signals from different regions of a user.

Embodiment 47. A method, comprising: disposing a portion of conductive segments onto a substrate; encapsulating the portion of conductive segments in a first portion of a flexible material such that the conductive segments are present at a first surface of the portion of the first portion of the flexible material; contacting the conductive segments with a current carrier; and disposing a second portion of flexible material so as to encapsulate the current carrier within the first portion of flexible material and the second portion of flexible material.

The methods can also include bending the first portion of flexible material, e.g., as shown in FIGS. 13B-13C. As shown figure, portion 1304 of flexible material can be bent using a stepped stage (e.g., a substrate that includes a depression) or other non-planar stage. The bending can be performed so as to give rise to a relatively higher portion (1320a) of the first surface and a relatively lower portion (1320b) of the first surface.

The current carrier can be contacted to the conductive segments at the relatively lower portion of the first surface, although this is not a requirement. The second portion of flexible material can be applied so as to fill in above the relatively lower portion of the first surface, as shown by the area between 1320a and 1320b in FIG. 13C.

It should be understood that the application of portion 1304c to portion 1304b can result in a monolithic portion in which no seam or interface between the portions is present, but this is not a requirement, as there can be an interface between portions of the flexible material. Thus, the current carrier can be encapsulated within a single, continuous portion of flexible material, but can also be encapsulated between two portions of flexible material, which portions are still considered part of the overall flexible material.

Embodiment 48. A sensor unit, comprising: a flexible material having disposed therein an amount of conductive segments, the conductive segments being present at a surface of the flexible material; a current carrier, the current carrier being in electrical communication with the conductive segments and the current carrier being at least partially encapsulated within the flexible material.

The flexible material can define a uniform thickness, but this is not a requirement, as the flexible material can define a first region having a first thickness and a second region having a second thickness that differs from the first thickness. The thickness that underlies a region where conductive segments are exposed for contact to a subject can be less than the thickness of a region where conductive segments are not exposed for contact to a subject. This is shown in FIG. 13C, in which thickness T1 is less than thickness T2.

As shown in FIGS. 13A-13C, a sensor unit can include a portion of conductive segments arranged such that some of the portion of conductive segments is exposed at a surface of the sensor unit, but another further portion of the conductive segments is disposed within the sensor unit, and can even be arranged such that the further portion of conductive segments is within the flexible material. The further portion of the conductive segments can be separated by a distance (measured along a surface of the sensor unit) from the exposed portion of conductive segments, e.g., a portion of conductive segments closer to a first end of the sensor unit is exposed, and a portion of conductive segments closer to a second end of the sensor unit is disposed within the flexible material.

Embodiment 49. A sensor unit, comprising: a flexible sensing layer comprising a plurality of conductive segments disposed in a first matrix material, the flexible sensing layer having a first surface configured for placement against a subject, the plurality of conductive segments optionally being characterized as a network, the plurality of conductive segments defining a first region of conductive segments that is exposed at the first surface, the plurality of conductive segments defining a second region of conductive segments that is enclosed within the flexible sensing layer, the second region of conductive segments being located at a distance, measuring along the first surface, from the first region of conductive segments; a current carrier in electronic communication with the conductive segments, the current carrier encapsulated within the flexible sensing layer, the current carrier being configured to carry a current to, from, or to and from the conductive segments.

Embodiment 50. The sensor unit of Embodiment 49, wherein the current carrier is enclosed within a first portion of the first matrix material and a second portion of the first matrix material.

Embodiment 51. The sensor unit of anyone of Embodiments 49-50, wherein the current carrier is characterized as being a wire.

Embodiment 52. The sensor unit of any one of Embodiments 59-51, wherein the conductive segments comprise one or more metals, graphene, carbon nanotubes, or any combination thereof.

Embodiment 53. The sensor unit of any one of Embodiments 49-53, wherein the conductive segments comprise one or more metals.

Embodiment 54. The sensor unit of any one of Embodiments 49-53, wherein the conductive segments comprise one or more metallic nanowires.

Embodiment 55. The sensor unit of Embodiment 54, wherein the conductive segments comprise gold nanowires, silver nanowires, or any combination thereof.

Embodiment 56. The sensor unit of any one of Embodiments 49-55, wherein the flexible sensing layer defines a region having a first thickness and a region having a second thickness.

Embodiment 57. The sensor unit of Embodiment 56, wherein the region having the first thickness underlies the first region of conductive segments of the plurality of conductive segments.

Embodiment 58. The sensor unit of any one of Embodiments 56-57, wherein the region having the second thickness underlies the second region of conductive segments of the plurality of conductive segments.

Embodiment 59. The sensor unit of any one of Embodiments 49-58, wherein at least some of the conductive segments have a salt coating disposed thereon.

Embodiment 60. The sensor unit of any one of Embodiments 49-59, wherein the flexible sensing layer defines one or more protrusions extending therefrom.

Embodiment 61. The sensor unit of any one of Embodiments 49-60, wherein the sensor unit is capable of maintaining electrical contact with a subject's skin in the absence of an electrolytic material disposed between the flexible sensing layer of the sensor unit and the subject's skin, wherein the sensor unit's collection of an electrical signal from a subject is substantially unaffected by movement artifacts, in the absence of an electrolytic material disposed between the flexible sensing layer of the sensor unit and the subject's skin, wherein noise in the sensor unit's collection of an electrical signal from a subject is substantially unaffected by movement artifacts, in the absence of an electrolytic material disposed between the flexible sensing layer of the sensor unit and the subject's skin, or wherein the sensor is capable of maintaining electrical contact with a subject's skin at a hairy region of the scalp, in the absence of an electrolytic material disposed between the flexible sensing layer of the sensor unit and the subject's skin.

Embodiment 62. A method, comprising collecting a signal from a subject with a sensor unit according to any one of Embodiments 49-62.

Embodiment 63. A method, comprising: disposing a portion of conductive segments onto a substrate; encapsulating the portion of conductive segments in a first portion of a flexible material such that the conductive segments are present at a first surface of the portion of the first portion of the flexible material; contacting the conductive segments with a current carrier; and disposing a second portion of flexible material so as to encapsulate the current carrier within the first portion of flexible material and the second portion of flexible material.

Embodiment 64. The method of Embodiment 63, further comprising stretching or wrinkling the first portion of flexible material.

Embodiment 65. The method of any one of Embodiments 63-64, wherein the conductive segments are metallic nanowires.

Embodiment 66. The method of any one of Embodiments 63-65, further comprising bending the first portion of flexible material so as to give rise to a relatively higher portion of the first surface and a relatively lower portion of the first surface.

Embodiment 67. The method of Embodiment 66, wherein the second portion of flexible material is disposed on the relatively lower portion of the first surface so as to encapsulate the current carrier between the first surface and the second portion of flexible material.

Embodiment 68. The method of any one of Embodiments 63-67, further comprising securing the current carrier to the plurality of conductive segments.

The present disclosure also contemplates methods of utilizing the disclosed devices. As one example, one can, based on a signal or signals collected by a device according to the present disclosure, alter (and/or initiate and/or cease) a stimulus that is administered to a subject being monitored by the device. As an example, one can alter and/or initiate and/or cease content (music, advertising, educational materials) that is delivered to a user based on a signal collected from the user during (or before or after) exposure to the content.

A subject can also utilize a device according to the present disclosure to modify their own activity. For example, a subject can monitor a figure of merit (e.g., engagement) while engaged in an activity, and alter their own activity (physical and/or mental) to affect that figure of merit while engaged in the activity. As another example, the disclosed technology can be used for fatigue monitoring, including real-time fatigue monitoring. The disclosed technology can further be used for stress monitoring, including real-time stress monitoring.

For any of the foregoing applications, a user can alter, remove, or add a task or other obligation depending on results collected using the disclosed technology. As one example, a user who is seen to experience a fatigue at or above a certain threshold level might be relieved of one or more tasks, e.g., a train dispatcher who is seen to experience fatigue may be replaced by a “fresh” dispatcher. In this way, a user can adjust work conditions and tasks, in real-time, based on the engagement, stress, and/or fatigue of workers.

### REFERENCES

Bertrand, A., Mihajlović, V., Grundlehner, B., Van Hoof, C. & Moonen, M. Motion artifact reduction in EEG recordings using multi-channel contact impedance measurements. In 2013 IEEE Biomedical Circuits and Systems Conference, BioCAS 2013 (2013). doi:10.1109/BioCAS.2013.6679688
Ratti, E., Waninger, S., Berka, C., Ruffini, G. & Verma, A. Comparison of Medical and Consumer Wireless EEG Systems for Use in Clinical Trials. Front. Hum. Neurosci. 11, (2017).
Hecht, D. S., Hu, L. & Irvin, G. Emerging transparent electrodes based on thin films of carbon nanotubes, graphene, and metallic nanostructures. Advanced Materials 23, 1482-1513 (2011).
Xu, F. & Zhu, Y. Highly conductive and stretchable silver nanowire conductors. Adv. Mater. 24, 5117-5122 (2012).
McClain, M. A. et al. Highly-compliant, microcable neuroelectrodes fabricated from thinfilm gold and PDMS. Biomed. Microdevices 13, 361-373 (2011).
Myers, A. C., Huang, H. & Zhu, Y. Wearable silver nanowire dry electrodes for electrophysiological sensing. RSC Adv. 5, 11627-11632 (2015).
www.cognionics.com/index.php/products/hd-eeg-systems/quick-20-dry-headset
www.choosemuse.com/
www.emotiv.com/epoc/
www.openbci.com/
www.sccn.ucsd.edu/eeglab/index.php

What is claimed:

1. A sensor unit, comprising:
a flexible sensing layer comprising a plurality of conductive segments disposed in a first matrix material,
the flexible sensing layer having a first surface configured for placement against a subject,
the plurality of conductive segments optionally being characterized as a network,
the plurality of conductive segments defining a first region of conductive segments that is exposed at the first surface,
the plurality of conductive segments defining a second region of conductive segments that is enclosed within the flexible sensing layer, the second region of conductive segments being located at a distance, measuring along the first surface, from the first region of conductive segments;
a current carrier in electronic communication with the conductive segments, the current carrier encapsulated within the flexible sensing layer,
the current carrier being configured to carry a current to, from, or to and from the conductive segments.

2. The sensor unit of claim 1, wherein the current carrier is enclosed within a first portion of the first matrix material and a second portion of the first matrix material.

3. The sensor unit of claim 1, wherein the current carrier is characterized as being a wire.

4. The sensor unit of claim 1, wherein the conductive segments comprise one or more metals, graphene, carbon nanotubes, or any combination thereof.

5. The sensor unit of claim 4, wherein the conductive segments comprise one or more metals.

6. The sensor unit of claim 5, wherein the conductive segments comprise one or more metallic nanowires.

7. The sensor unit of claim 6, wherein the conductive segments comprise gold nanowires, silver nanowires, or any combination thereof.

8. The sensor unit of claim 1, wherein the flexible sensing layer defines a region having a first thickness and a region having a second thickness.

9. The sensor unit of claim 8, wherein the region having the first thickness underlies the first region of conductive segments of the plurality of conductive segments.

10. The sensor unit of claim 9, wherein the region having the second thickness underlies the second region of conductive segments of the plurality of conductive segments.

11. The sensor unit of any claim 1, wherein at least some of the conductive segments have a salt coating disposed thereon.

12. The sensor unit of claim 1, wherein the flexible sensing layer defines one or more protrusions extending therefrom.

13. The sensor unit of claim 1, wherein the sensor unit is capable of maintaining electrical contact with a subject's skin in the absence of an electrolytic material disposed between the flexible sensing layer of the sensor unit and the subject's skin, wherein the sensor unit's collection of an electrical signal from a subject is substantially unaffected by movement artifacts, in the absence of an electrolytic material disposed between the flexible sensing layer of the sensor unit and the subject's skin, wherein noise in the sensor unit's collection of an electrical signal from a subject is substantially unaffected by movement artifacts, in the absence of an electrolytic material disposed between the flexible sensing layer of the sensor unit and the subject's skin, or wherein the sensor is capable of maintaining electrical contact with a subject's skin at a hairy region of the scalp, in the absence of an electrolytic material disposed between the flexible sensing layer of the sensor unit and the subject's skin.

14. A method, comprising collecting a signal from a subject with a sensor unit according to claim 1.

15. A method, comprising: disposing a portion of conductive segments onto a substrate; encapsulating the portion of conductive segments in a first portion of a flexible material such that the conductive segments are present at a first surface of the portion of the first portion of the flexible material; contacting the conductive segments with a current carrier; and disposing a second portion of flexible material so as to encapsulate the current carrier within the first portion of flexible material and the second portion of flexible material.

16. The method of claim 15, further comprising stretching or wrinkling the first portion of flexible material.

17. The method of claim 15, wherein the conductive segments are metallic nanowires.

18. The method of claim 15, further comprising bending the first portion of flexible material so as to give rise to a relatively higher portion of the first surface and a relatively lower portion of the first surface.

19. The method of claim 18, wherein the second portion of flexible material is disposed on the relatively lower portion of the first surface so as to encapsulate the current carrier between the first surface and the second portion of flexible material.

20. The method of claim 18, further comprising securing the current carrier to the plurality of conductive segments.
